(19) United States
(12) Patent Application Publication
(10) Pub. No.: US 2004/0254461 A1
(43) Pub. Date: Dec. 16, 2004

(54) Acoustic beam shaping by pulse power modulation at constant amplitude

(76) Inventor: William H. Ackerman III, Somerdale, NJ (US)

Correspondence Address: Ralph W. Selitto, Jr., McCarter & English, LLP, Four Gateway Center, 100 Mulberry Street, Newark, NJ, 07102, US

(21) Appl. No.: 10/765,013

(22) Filed: Jan. 26, 2004

Related U.S. Application Data
(63) Continuation-in-part of application No. 09/926,666, filed on Mar. 20, 2002, now Pat. No. 6,682,483.
Application No. 09/926,666 is a 371 of international application No. PCT/US00/14691, filed on May 26, 2000.

(51) Int. Cl. A61B 8/00

(52) U.S. Cl. 600/437; 600/459

(57) ABSTRACT

In traditional ultrasound applications the transmit signal is a short series of constant amplitude pulses. The pulses are 50 percent duty cycle and constitute a single frequency. The amplitude must be very consistent or system performance will suffer. Modern ultrasound requires shaping the transmitter beam by applying different amplitudes to an array of elements. However, the need to change voltage from one series to the next can cause problems with the electronics associated with the transmission. One problem is there is not enough time for the voltage to settle from one series to the next. This has the effect of causing artifacts in the image. One means of overcoming the problems is to hold the voltage constant and modify the modulation of the series of pulses to achieve the different power levels.

### PRIORITY

This application is a CIP of U.S. application Ser. No. 09/926,666, filed Nov. 30, 2001 and scheduled to issue on Jan. 27, 2004 as U.S. Pat. No. 6,682,483 and depends for priority on U.S. Provisional Application No. 60/446,162, filed Feb. 10, 2003. application Ser. No. 09/926,666 depends for priority on international application PCT/US00/14691, filed May 26, 2000. The International application depends for priority on U.S. Provisional Applications Nos. 60/136,364 filed May 28, 1999, 60/138,793 filed Jun. 14, 1999 and 60/152,886 filed Sep. 8, 1999.

### BACKGROUND OF THE INVENTION

1. Field of the Invention

This invention relates to ultrasonic acoustic imaging, primarily for medical purposes.

2. Brief Description of the Background Art

Ultrasonic acoustic imaging finds many uses, particularly in the field of non-invasive medical testing. Direct detection of the emitted acoustic frequency permits, for example, prenatal fetal imaging. Detection of Doppler shifted acoustic frequencies permits observation of flow of a particle-containing liquid, for example, blood flow. Acoustic imaging equipment utilizes a probe that is applied to the skin of the patient overlying the part of the body being investigated. At the end of the probe is an array of transducers, usually piezoelectric, that are excited by bursts of electrical energy at the ultrasonic frequency and, for example, square wave modulated to transmit square wave modulated bursts of ultrasonic energy into the body region being investigated. The subsurface structures reflect some of that energy, either at the transmitted frequency or Doppler shifted, back to the probe, where it is detected by piezoelectric receiver elements in the probe.

One application of this technology to the three dimensional mapping and tracking of blood flow is disclosed in parent U.S. application Ser. No. 09/926,666, which is scheduled to issue on Jan. 27, 2004 as U.S. Pat. No. 6,682,483. The pertinent text of that application is included herein below.

### SUMMARY OF THE INVENTION

In traditional ultrasound applications the transmit signal is a short series of constant amplitude pulses. The pulses are 50 percent duty cycle pulse of acoustic energy at a single frequency. The amplitude must be very consistent or system performance will suffer. More advanced ultrasound techniques require shaping the transmitted beam by applying different power levels to different elements of an array of transmitter elements. However, the need to change voltage from one series of pulses to the next can cause problems with the electronics associated with the transmission. One problem is there is not enough time for the voltage to settle from one series to the next. This has the effect of causing artifacts in the image.

One means of overcoming the problems is to hold the voltage constant and modify a series of pulses to achieve the different power levels. Two techniques can be used with the pulses to control the power. The first is using fewer pulses in the series and the second is modifying the width of the pulses, thus, pulse width modulation. When using fewer pulses, the start of the series is delayed to position the center of series in the correct relationship with the other series. This invention employs both techniques. By varying both number and width a wide range of power levels can be achieved. The bandwidth characteristic of the piezoelectric element and system smoothes the pulse width modulated signal into a lower amplitude signal with greatly reduced harmonics. A second benefit is the reduced complexity of the transmit circuitry. Without this invention a significant amount of high power low impedance circuitry is required to rapidly change and hold the voltage at a new level. To do this with precision adds even more circuitry. The amount of digital logic necessary to create this new pulse width modulated transmit signal is small and does not significantly affect the cost of the system.

### BRIEF DESCRIPTION OF THE DRAWINGS

FIG. 24A illustrates a series of control signals for transmitting a 50 percent duty cycle burst of ultrasonic energy at a reference amplitude.

FIG. 24B illustrates a series of control signals that implement the prior art method of reducing the burst of energy by reducing the amplitude of he control signal.

FIG. 24C illustrates a series of control signals that implement the herein disclosed method of reducing the burst energy by reducing the width of the pluses to significantly less than 50 percent duty cycle.

FIG. 24D illustrates a series of control signals that product a reduction of bursts of energy by also reducing the number of pulses in the burst, while keeping the center of the burst in correct temporal relationship with other bursts.

FIG. 25 is a circuit diagram of an exemplary apparatus for controlling and transmitting a series of bursts of varying amplitude.

FIG. 26 is a circuit diagram of an exemplary transmission apparatus for varying burst of power by controlling pulse width and/or number of pluses per burst.

### DETAILED DESCRIPTION OF THE INVENTION

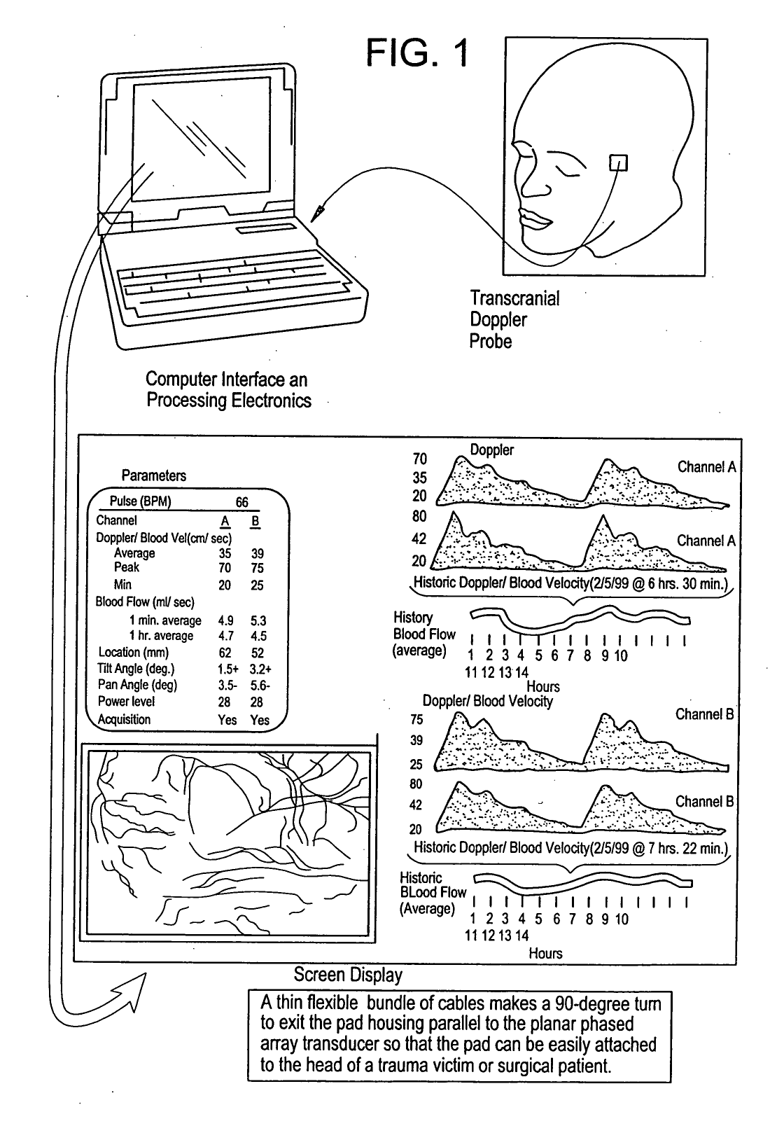

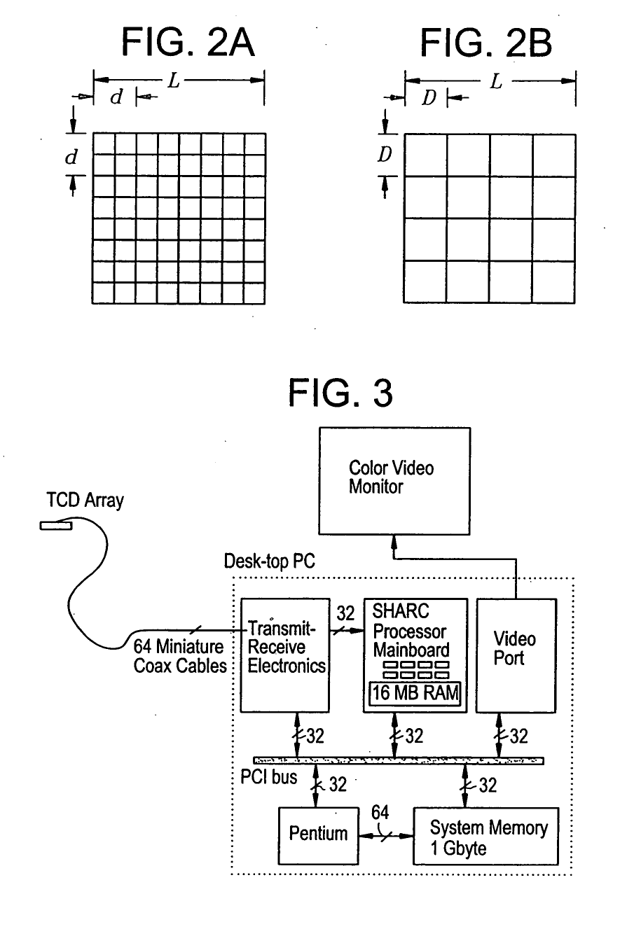

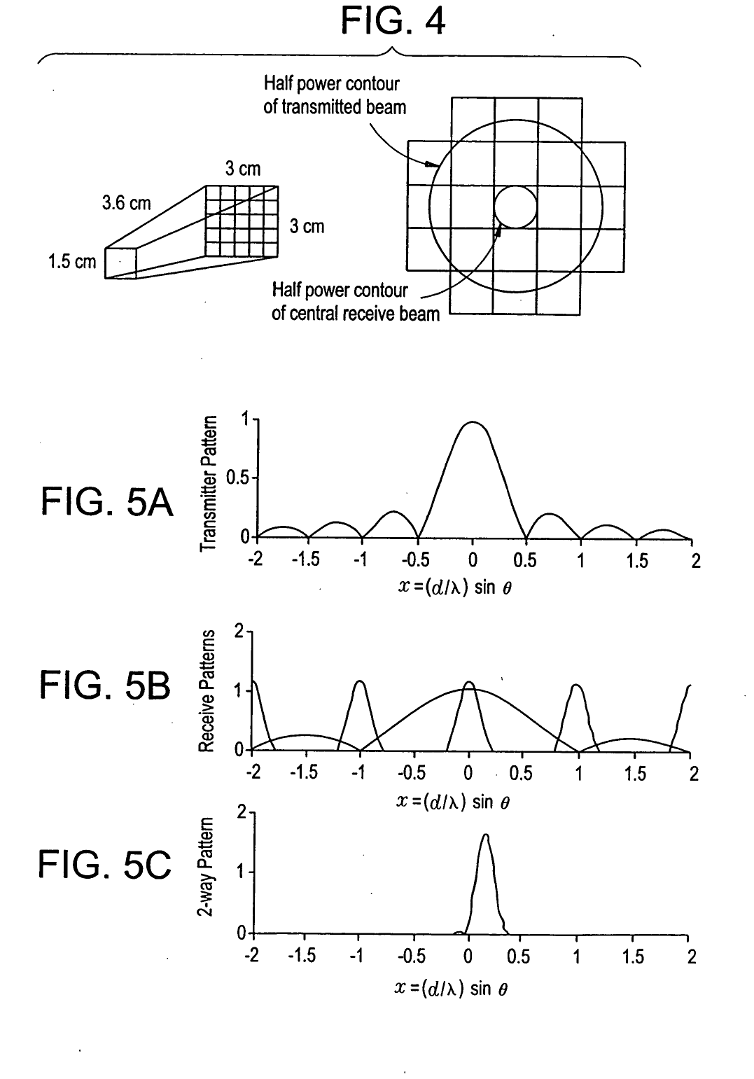

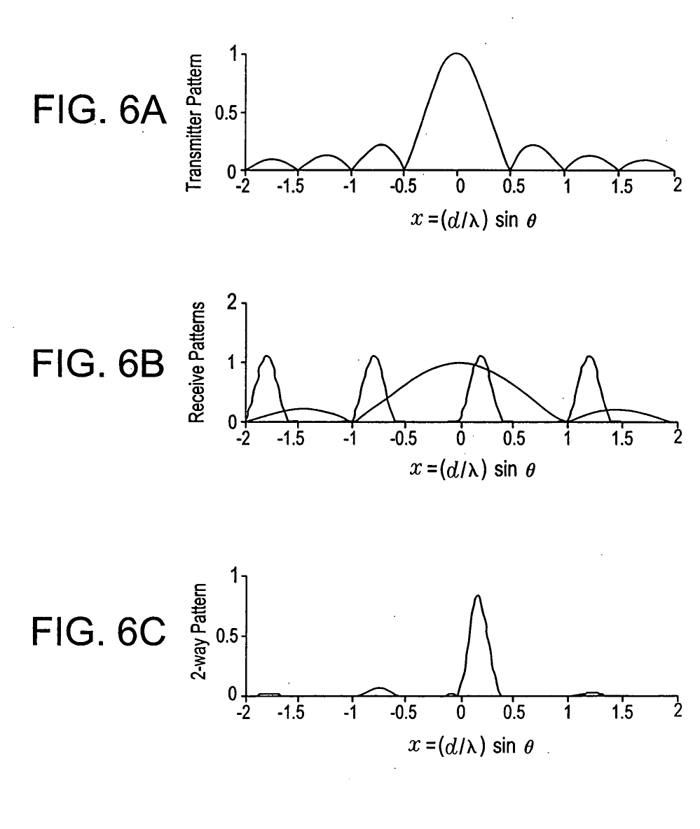

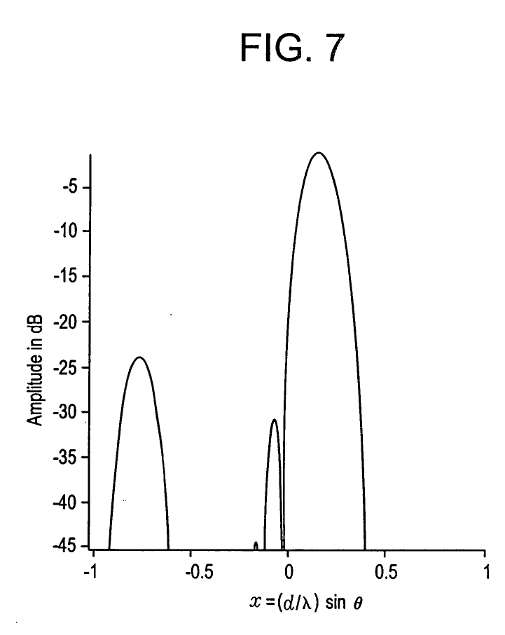

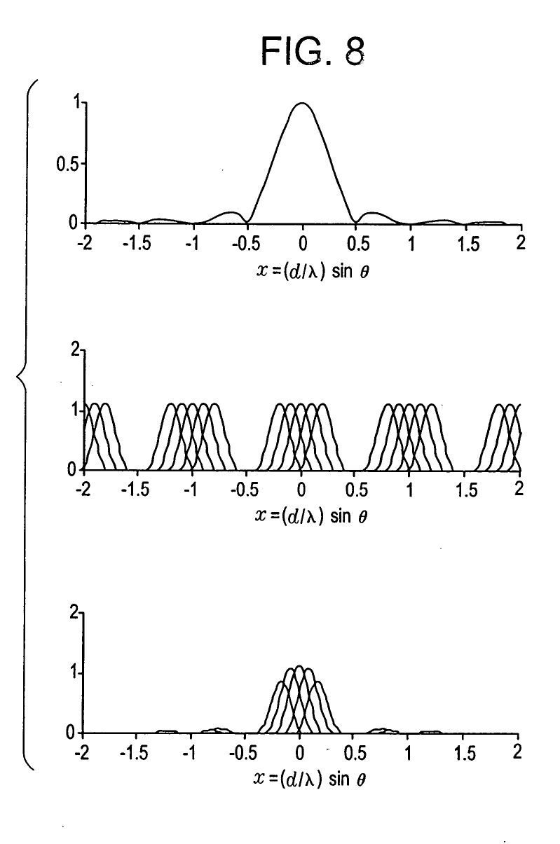

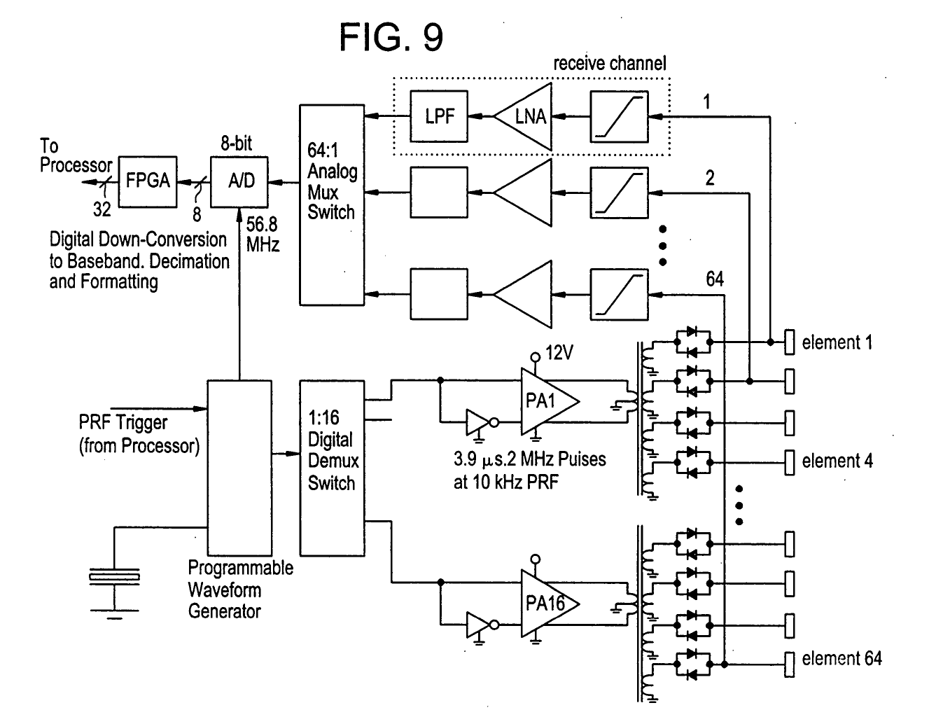

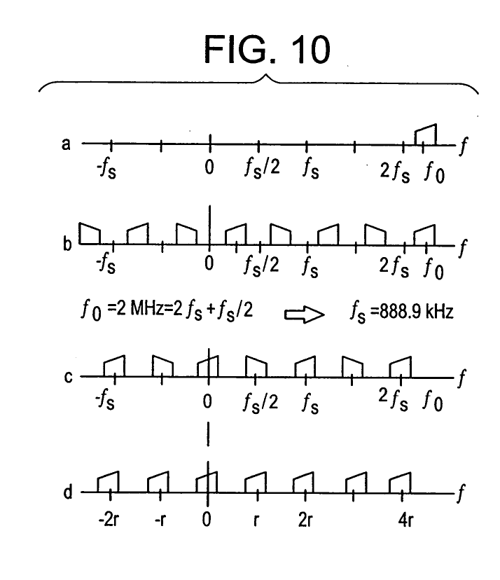

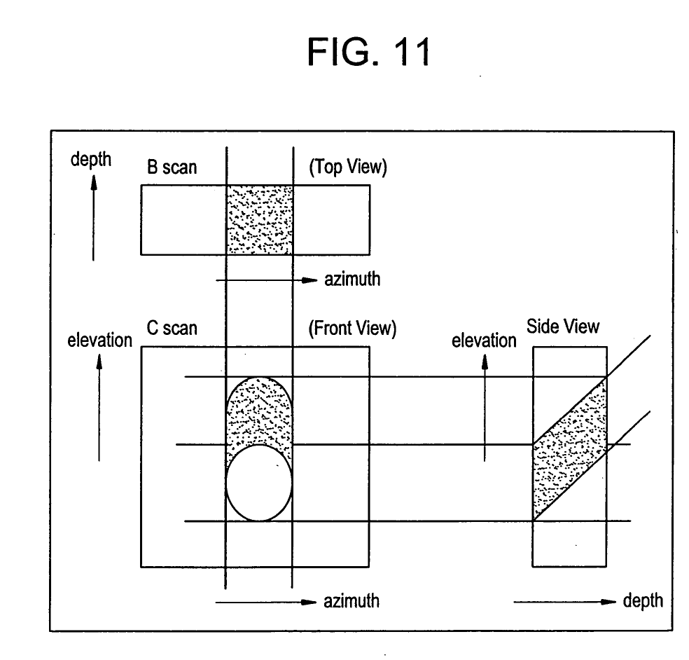

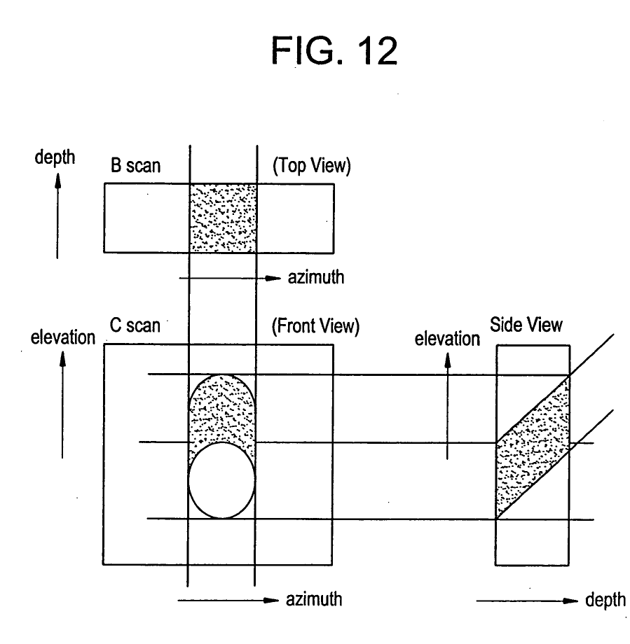

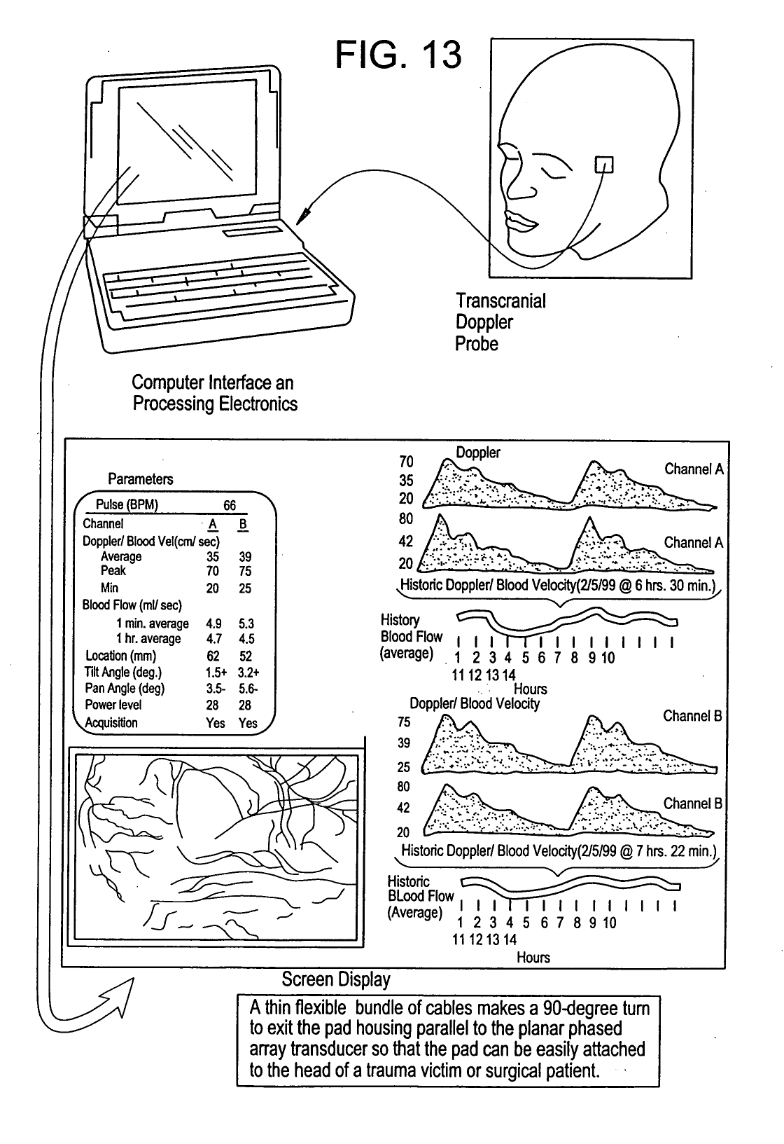

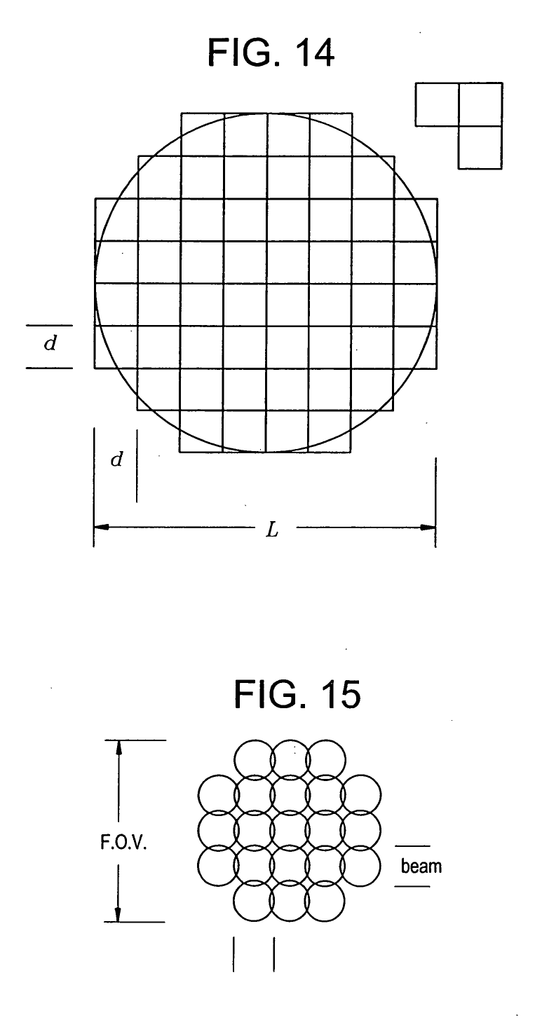

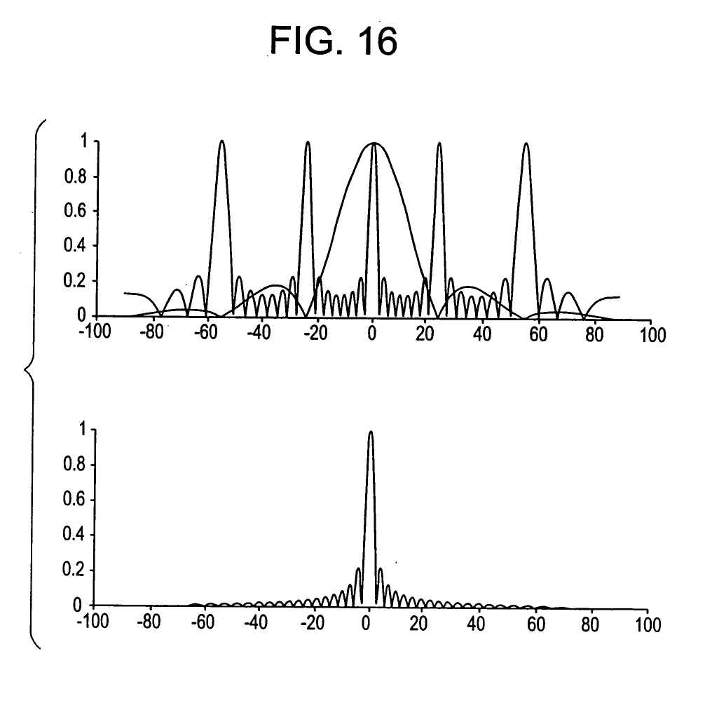

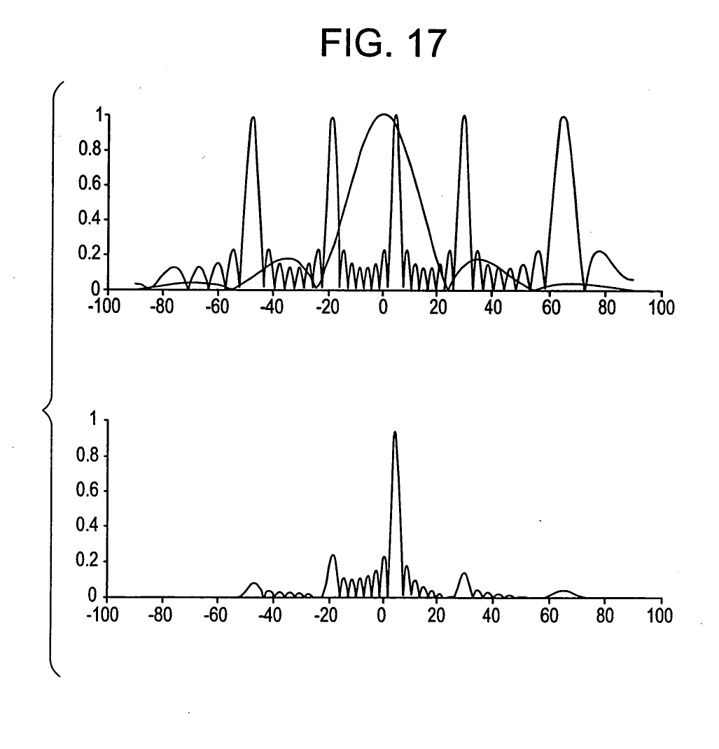

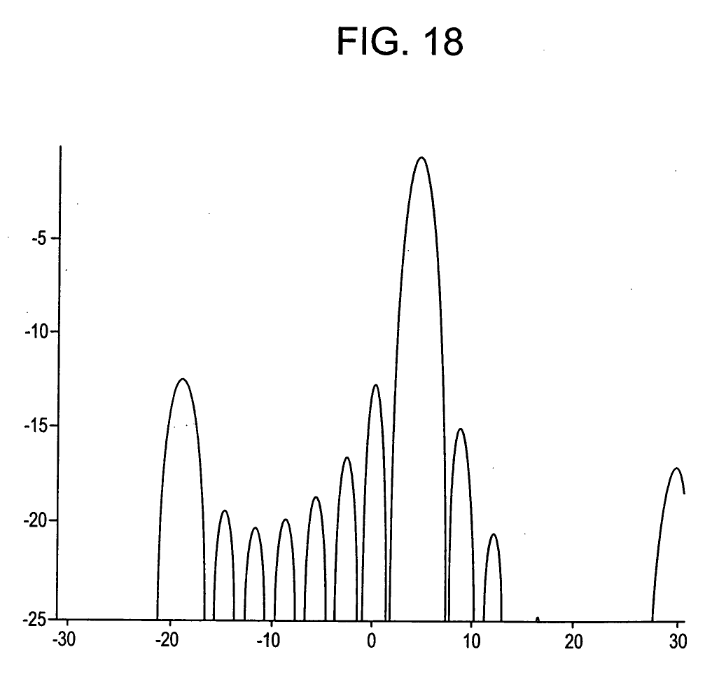

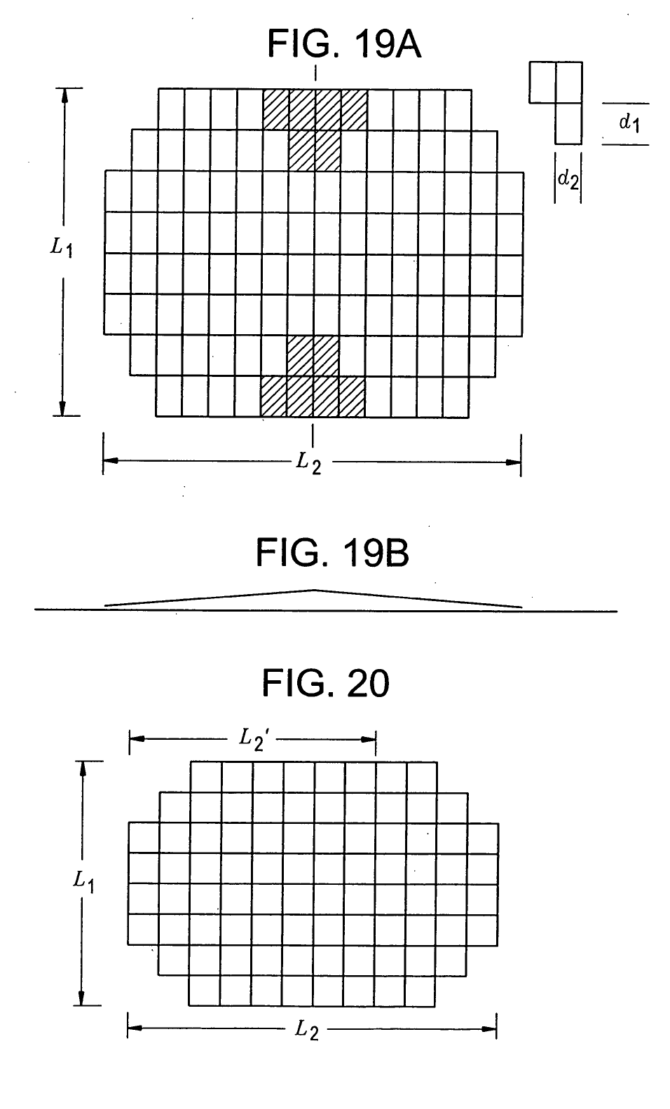

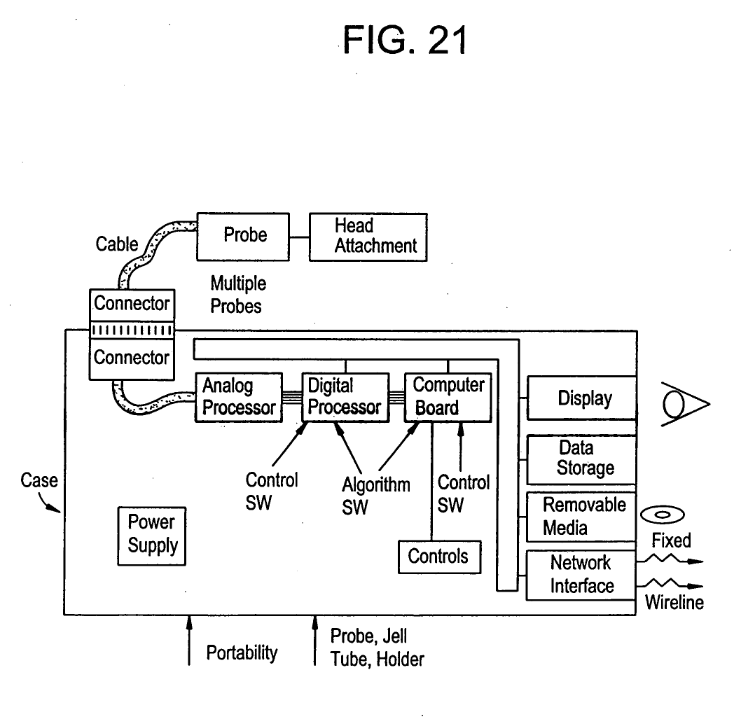

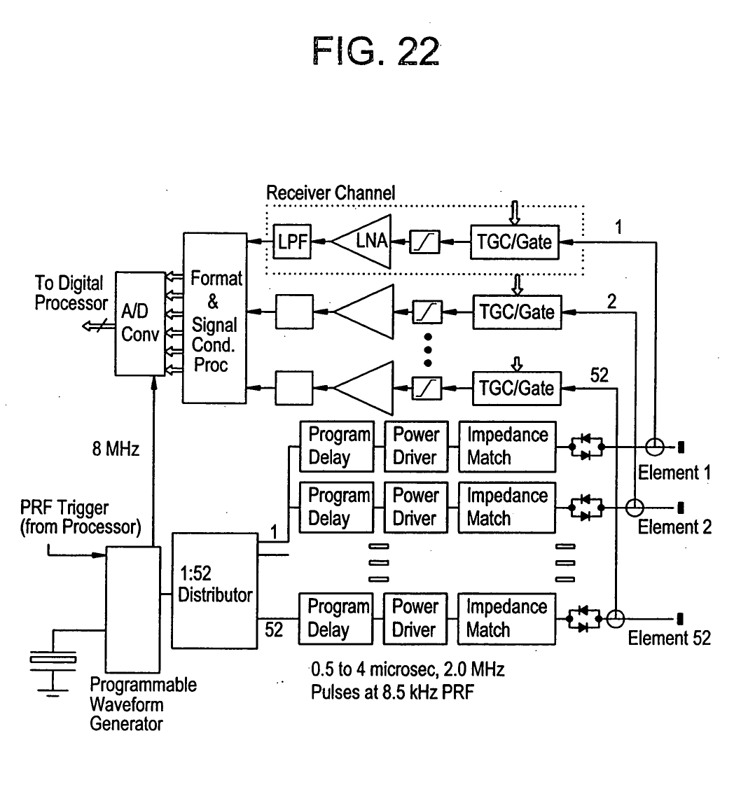

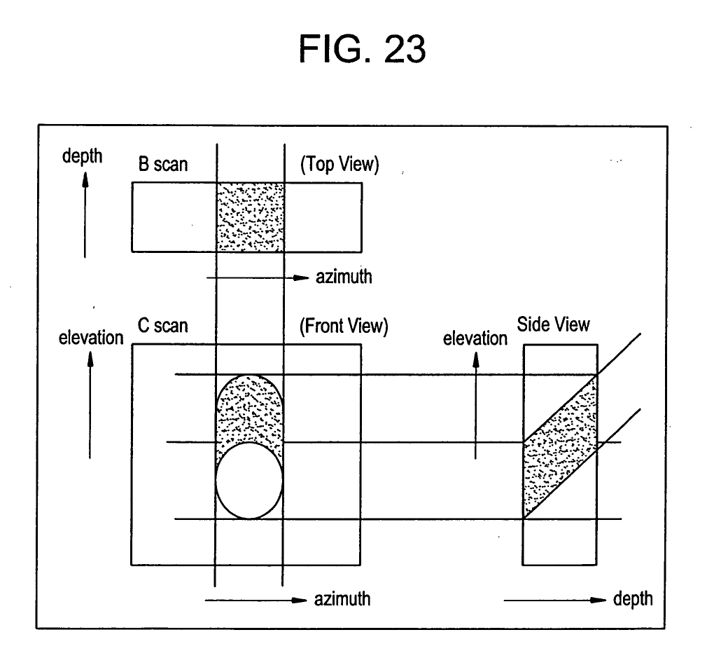

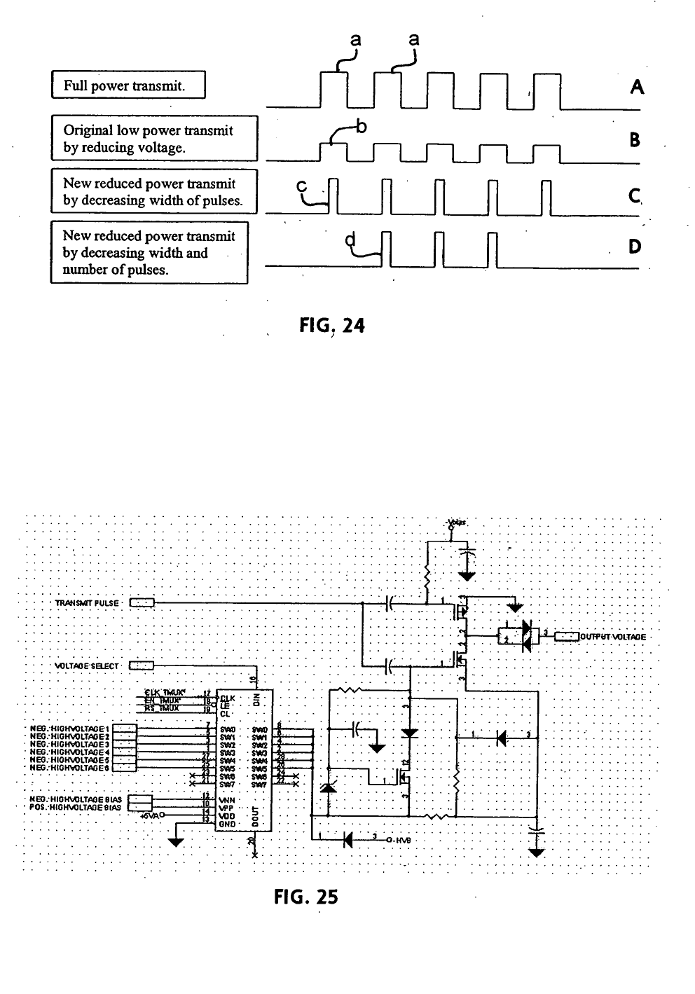

FIGS. 24A-24D represent four different excitations of one transmitter transducer element of an ultrasonic probe. Each square wave (a, b, c, d) represents a constant amplitude pulse of ultrasonic excitation of defined pulse width. The series of five pulses illustrated in FIG. 24A will transmit a burst of acoustic energy into the patient at a defined power level. It has been found that, in order to reduce the level of side lobe transmission, it is desirable to excite probe elements at the edges of the probe (See, for example, FIG. 2) at a lower power level than elements at the center of the probe. In the prior art this is done by reducing the amplitude of transducer element excitation, as illustrated in FIG. 24B.

In the inventive method described herein, the transmitted power is reduced by reducing the pulse width, as illustrated in FIG. 24C or by reducing the number of pulses in each burst, as illustrated in FIG. 24D. The reduced number of pulses can be at a reduced probe width to achieve a wide range of power level control, as illustrated in FIG. 24D, or at the full pulse width, which may be easier to implement electronically.

The amount of power reduction at the probe periphery should be sufficient to produce an operationally significant reduction of harmonics in the modulated signal.

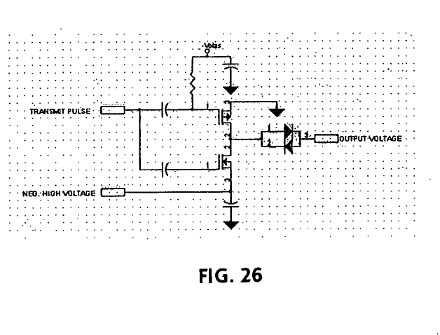

FIG. 25 illustrates exemplary circuitry used to excite probe elements at variable amplitude and pulse count. FIG. 26 illustrates the much simpler circuitry with which probe elements are excited at constant amplitude with variable pulse width and variable number of pulses.

The disclosure that follows illustrates the exemplary use of Doppler shifted acoustic imaging to map and track blood flow. It illustrates the techniques and systems that profit from application of the hereindisclosed invention. Alternative probe geometries to which the hereindisclosed invention could be profitably applied are illustrated in U.S. Pat. No. 6,524,253 B1, which is incorporated by reference in its entirety herein.

What is claimed is:

1. A method for transmitting a plurality of short series of bursts of ultrasonic energy pulses from a probe terminated by an array of transducer elements; and modulating an amount of energy transmitted by each burst from transducer element to transducer element across the probe by varying the width of each energy pulse or varying the number of pulses per burst or varying both the width of each pulse and the number of pulses per burst while keeping each pulse amplitude constant from pulse to pulse across the probe, the modulating being sufficient to produce an operationally significant reduction of harmonics in the modulated signal.

2. The method of claim 1 in which the amount of energy transmitted by each burst is less at the array's edge than at the array's center.

3. The method of claim 2 in which the amount of energy transmitted by each burst is progressively reduced from a maximum at the array's center to a minimum at the array's edge.

4. The method of claim 2 in which the array is a one dimensional array or a two dimensional array.

5. An apparatus for transmitting a plurality of short series of bursts of ultrasonic energy pulses from a probe terminated by a one dimensional or a two dimensional array of transducer elements and modulating an amount of energy transmitted by each burst from transducer element to transducer element across the probe, comprising control circuitry incorporating digital logic adapted for varying the width of each pulse or the number of pulses per burst or both the width of each pulse and the number of pulses per burst from burst to burst across the probe, the modulating being sufficient to produce an operationally significant reduction of harmonics in the modulated signal.

6. An apparatus of claim 5 in which the amount of energy transmitted by each burst is less at the array's edge than at the array's center.

7. An apparatus of claim 6 in which the amount of energy transmitted by each burst is progressively reduced from a maximum at the array's center to a minimum at the array's edge.
